(19) United States
(12) Patent Application Publication
(10) Pub. No.: US 2026/0283958 A1
(43) Pub. Date: Sep. 24, 2026

(54) FORMULATIONS OF MICROPARTICLES INCLUDING HYALURONIDASE

(71) Applicant: Serán BioScience, LLC, Bend, OR (US)

(72) Inventors: Erica Schlesinger, Sisters, OR (US); Cindy Chung, Bend, OR (US); Dan Smithey, Bend, OR (US)

(21) Appl. No.: 19/572,806

(22) Filed: Mar. 19, 2026

Related U.S. Application Data
(60) Provisional application No. 63/774,653, filed on Mar. 19, 2025.

(51) Int. Cl. A61K 9/16; A61K 31/20

(52) U.S. Cl. CPC A61K 9/1658; A61K 9/1694; A61K 31/20

(57) ABSTRACT

A formulation is provided for a hyaluronidase enhanced microparticle formulation. The hyaluronidase microparticle formulation includes microparticles including an active pharmaceutical ingredient, hyaluronidase, and a non-aqueous vehicle.

### CROSS-REFERENCE TO RELATED APPLICATIONS

The present application claims priority to U.S. Provisional Application No. 63/774,653 entitled “FORMULATIONS OF MICROPARTICLES INCLUDING HYALURONIDASE” and filed on Mar. 19, 2025. The entire contents of the above listed application is hereby incorporated by reference for all purposes.

### FIELD

The present description relates generally to pharmaceutical formulations containing hyaluronidase and an active pharmaceutical or biopharmaceutical component in the form of solid state microparticles.

### BACKGROUND/SUMMARY

Subcutaneous injections are an attractive drug delivery route, especially for active pharmaceutical ingredients (APIs) which may not be suited for oral delivery. Subcutaneous injections result in a fluid bolus that, depending on the formulation and injection volume, may take a significant time to disperse. A slow dispersion rate of the bolus upon injection may negatively impact injection tolerability and the overall patient experience. Further, slow dispersion may slow absorption and decrease bioavailability of the API. Subcutaneous injections of formulations comprising a high concentration suspension of microparticles in a non-aqueous vehicle may be preferred over an aqueous injection. The high concentration suspension of microparticles provides the ability to deliver a high dose of API in small dose volumes (e.g., 1 mL to 3 mL) that otherwise demands high dose volumes and potentially long infusion times for the same API dose provided as an aqueous solution in which concentrations are limited to typically less than 150 mg/mL. However, dispersion times for the suspension may be slow for such injections due to the high viscosity and poor miscibility of the non-aqueous carrier in the aqueous cellular environment.

Hyaluronidase is an enzyme which breaks down hyaluronan, a component of the extracellular matrix. Conventionally, hyaluronidase may be included in an aqueous injection to help break down the extracellular matrix and enable a more rapid dispersion of the injected fluid. In this way, the hyaluronidase provides more space for an injected bolus to disperse and is often referred to as a permeation enhancer. Unlike dissolution enhancers, such as surfactants, which increase the miscibility of the injection vehicle with the physiological fluid, hyaluronidase works regardless of the miscibility of the injected material with the physiological fluid. Hyaluronidase is most commonly integrated into pharmaceutical products either as a co-formulation or in a co-dosing regimen with large volume subcutaneous delivery of solutions. The use of hyaluronidase to aid in administration of suspension formulations has not previously been considered. Co-delivery of hyaluronidase with a suspension could either enable delivery of increased suspension volumes, increased rates of administration, or improvements in tolerability and patient experience. However, as a water soluble protein, incorporation of hyaluronidase into these non-aqueous suspensions is non-trivial.

The inventors have recognized the above issues and in one example, the issues described above may be at least partially addressed by a non-aqueous formulation including both the hyaluronidase and the suspended microparticles. For example, a hyaluronidase enhanced microparticle suspension formulation comprises: microparticles including an active pharmaceutical ingredient, hyaluronidase, and a non-aqueous vehicle. The hyaluronidase microparticle formulation may improve both patient experience and outcomes by enabling co-formulation of hyaluronidase with a therapeutic protein in a high concentration suspension, combining the high dose advantages of a microparticle suspension with the known activity of hyaluronidase to break down the ECM, leading to more rapid dispersion of a bolus subcutaneous injection.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows diagrams of examples of hyaluronidase enhanced microparticle suspension formulations representing the different mechanisms by which hyaluronidase can be co-formulated or co-delivered with an active pharmaceutical ingredient (API).

FIG. 2 shows a flow chart of an example of a method for preparing a first example of the hyaluronidase enhanced microparticle formulation.

FIG. 3 shows a flow chart of an example of a method for preparing a second example of the hyaluronidase solubilized microparticle formulation.

FIG. 4 shows a flow chart of an example of a method for preparing a third example of the hyaluronidase solubilized microparticle formulation.

FIG. 5 shows an example of the hyaluronidase enhanced microparticle formulation as a kit supporting co-delivery of a high concentration suspension with a hyaluronidase solution or suspension. In such a kit the hyaluronidase and microparticle formulation comprising the API are independently formulated.

FIG. 6 shows a chart comparing hyaluronidase activity in reconstituted co-processed spray dried particles containing hyaluronidase and a protein therapeutic to the activity of the hyaluronidase raw material.

FIG. 7 shows a chart comparing hyaluronidase activity in reconstituted spray dried particles containing hyaluronidase and excipients to the activity of the hyaluronidase raw material.

FIG. 8 shows a chart comparing hyaluronidase activity in co-processed, co-formulated and emulsion spray dried particle suspensions.

### DETAILED DESCRIPTION

The use of hyaluronidase in co-formulations with therapeutic proteins to improve patient experience through enhancing diffusion of a bolus dose within the extracellular matrix upon administration has been previously demonstrated in commercially available products such as those integrating Halozyme's ENHAZE® technology. It is understood that the benefit of decreased pain and increased rate of diffusion comes from the activity of hyaluronidase when co-delivered with a protein therapeutic. Conventionally, leveraging hyaluronidase in such a way is limited to solution formulations, which are limited in concentration of the protein therapeutic by stability and viscosity. To deliver therapeutic doses with these solution formulations, relatively large volumes are demanded; the delivery of these large volumes is enabled by the co-formulation with hyaluronidase. An alternative approach to subcutaneous delivery of high dose protein therapeutics is to leverage technologies that enable much higher concentrations of the protein therapeutic, allowing for high dose delivery in standard volumes for injection (≤2 mL). One example of such a technology is formulation of a high concentration suspension prepared from solid microparticles comprised of the therapeutic protein suspended in non-aqueous vehicles. The incorporation of hyaluronidase into these suspension formulations may provide additional benefit, enabling an improved patient experience (e.g., reduction in pain upon administration) for a standard volume or enabling even higher doses through delivery of larger volumes of suspension (>2 mL). However, while co-formulation of hyaluronidase with protein therapeutics in solution is relatively straightforward, the co-formulation of hyaluronidase in suspension has not previously been explored and could be approached in a variety of ways, with results that are not predictable a priori.

To leverage the benefit of hyaluronidase, it is delivered in a co-formulation in which the enzyme retains activity. Activity of the enzyme in the final product depends on the formulation and process for manufacturing; hyaluronidase is not conventionally formulated as a spray dried dispersion or in a non-aqueous suspension comprised of solid state microparticles. Furthermore, while trivial in the consideration of solution formulations, the drastic difference in dose for the hyaluronidase and protein therapeutic is considered in the selection of manufacturing technologies and product design for co-delivery of these two components as microparticles.

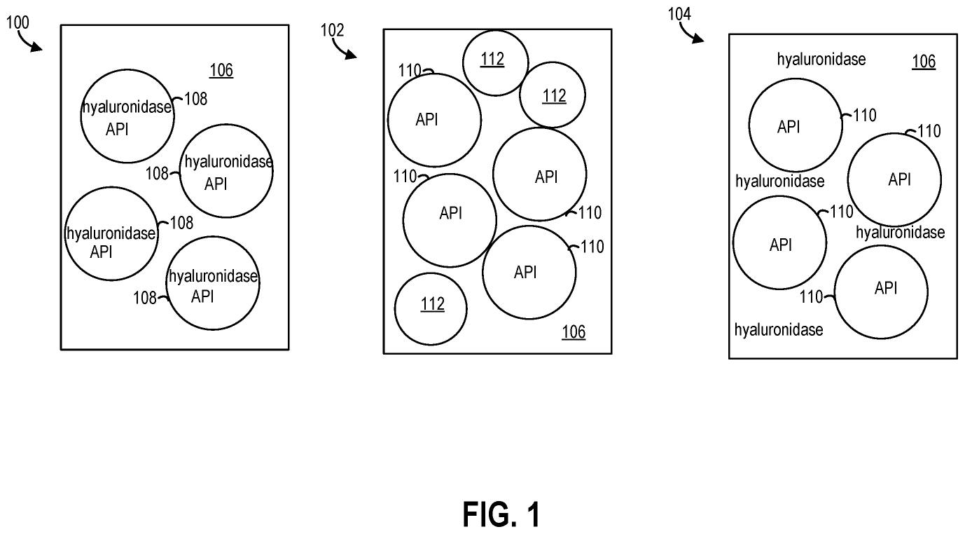

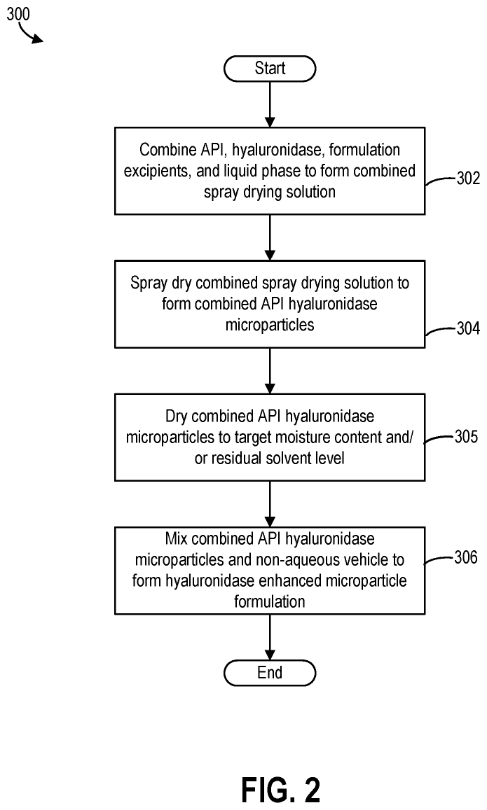

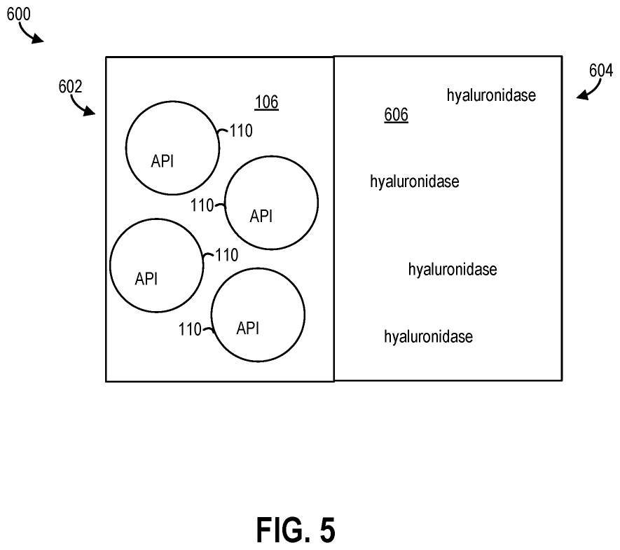

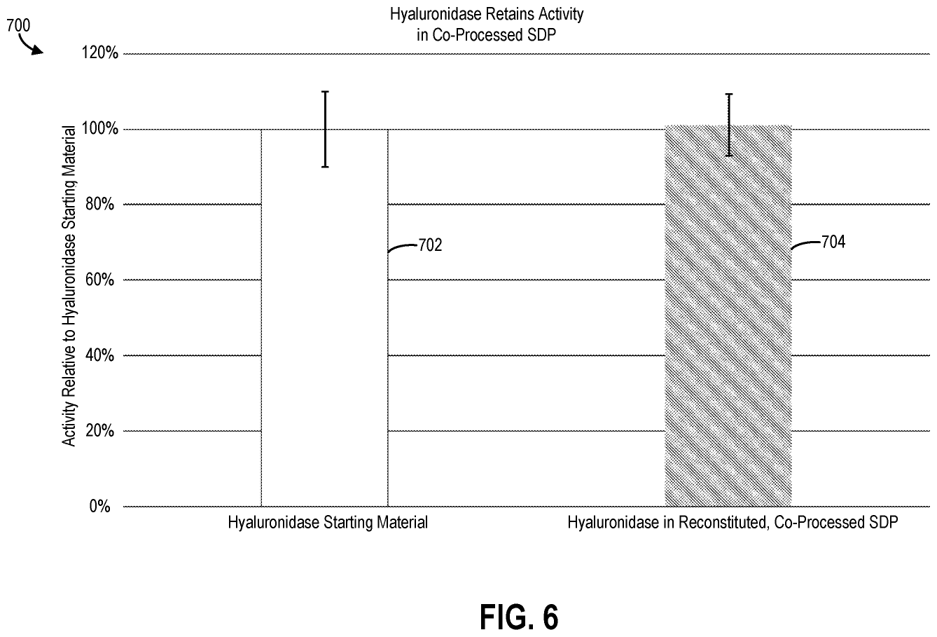

The following description relates to a formulation of microparticles in a non-aqueous vehicle including an active pharmaceutical ingredient (API) and hyaluronidase. The formulation may be herein referred to as a hyaluronidase enhanced microparticle suspension formulation. Examples of the hyaluronidase enhanced microparticle suspension formulations are shown pictorially in FIG. 1 and FIG. 5. Hyaluronidase may be incorporated into the microparticle with the API, into a microparticle separate from the API, or into the vehicle in which a microparticle containing API is suspended. Through the activity of hyaluronidase, the hyaluronidase enhanced microparticle suspension formulation may exhibit faster dispersion of the fluid upon injection thereby improving patient experience and providing enhanced absorption times and/or bioavailability compared to a microparticle suspension formulation which does not include hyaluronidase. In some examples, the microparticles of the hyaluronidase enhanced microparticle suspension formulation are spray dried microparticles. Methods of preparing different examples of the hyaluronidase enhanced microparticle suspension formulations using spray drying are shown in FIGS. 2-4. Examples of hyaluronidase enhanced microparticle formulations were prepared and tested for resulting hyaluronidase activity. The results are shown in FIG. 6-8.

It should be noted that while the examples given here illustrate the use of spray drying to produce microparticles for suspension, there are other equivalent techniques to produce microparticles for similar use, including precipitation. The hyaluronidase enhanced suspension formulations described herein regarding the incorporation of hyaluronidase into the microparticle containing an active or formulation of hyaluronidase as a microparticle for co-delivery with a microparticle containing an active relates to any standard or previously disclosed process for microparticle production.

A hyaluronidase enhanced microparticle suspension formulation, as described herein, may combine the increased rate of dispersion achieved through co-formulation of an active pharmaceutical ingredient with hyaluronidase in an aqueous formulation with the high concentration/small injection volume of a non-aqueous microparticle suspension. Turning now to FIG. 1, diagrams show a first example 100, a second example 102, and a third example 104 of hyaluronidase enhanced microparticle suspension formulations. Diagrams of examples 100, 102, and 104 are not shown to scale and do not represent relative sizes or concentrations of components of the hyaluronidase enhanced microparticle suspension formulations. Additionally, the hyaluronidase enhanced microparticle suspension formulations may include additional formulation excipients, such as, but not limited to, buffer salts, stabilizers, surfactants and preservatives which are not shown in diagrams of examples 100, 102, and 104. The hyaluronidase enhanced microparticle suspension formulation may include a non-aqueous vehicle, an API, and hyaluronidase. As shown in first example 100, the hyaluronidase may be co-formulated in a microparticle with the API 108, or as shown in second example 102, the hyaluronidase may be formulated independently as a microparticle and co-delivered in a non-aqueous vehicle 106 with microparticles containing an API. Third example 104 illustrates a third concept in which hyaluronidase is added directly to the non-aqueous vehicle 106 in which microparticles comprised of API are suspended. In this instance, hyaluronidase may remain solubilized in the vehicle or may precipitate upon addition.

In one example, the API may be a biologic having therapeutic properties. For example, the API may be one or more of, but not limited to, an oligonucleotide, a fraction of RNA or DNA, protein, peptide, enzyme or an antibody. The RNA or DNA may each be single stranded or double stranded and may include at least 10 bases. Types of RNA may include, but are not limited to, messenger RNA (mRNA), transfer RNA (tRNA), small interfering RNA (siRNA), microRNA (miRNA), RNA aptamers, and antisense RNA (asRNA). Types of DNA may include, but are not limited to, plasmid DNA, DNA aptamers, DNA acidzymes, antisense oligonucleotides, and antigene oligonucleotides. In alternate examples, the API may be a pharmaceutically active small molecule. In some examples, a concentration of the API in the hyaluronidase solubilized microparticle formulation may be greater than or equal to 150 mg/mL.

The hyaluronidase is an enzyme which breaks down hyaluronic acid. The hyaluronidase may cleave glycosidic bonds of the hyaluronic acid to degrade a structure of the hyaluronic acid. A source of the hyaluronidase may not be particularly limited. For example, the hyaluronidase may be mammalian and/or microbial in origin. In some examples, the hyaluronidase may be a recombinant hyaluronidase. Examples of therapeutically relevant and commercially available hyaluronidase are rhuP20 and ALT-B4.

The non-aqueous vehicle may be selected from Federal Drug Administration (FDA) approved vehicles based on a viscosity, density, and compatibility of the vehicle with the API, proteins, and injection device materials. As one example, the vehicle may be a mix of glyceryl tricaprylate/tricaprate, triacetin, ethyl oleate, or propylene glycol dicaprylate/dicaprate, combinations of the aforementioned vehicles, or other proprietary compositions, among others.

A first example 100 of the hyaluronidase enhanced microparticle suspension formulation includes a non-aqueous vehicle 106 and combined API hyaluronidase microparticles 108. First example 100 may be referred to as a co-processed suspension. Combined API hyaluronidase microparticles 108 may be microparticles which each include both the API and the hyaluronidase homogeneously distributed throughout each microparticle. Each combined API hyaluronidase microparticle 108 may include more API than hyaluronidase. The combined API hyaluronidase microparticles may be spray dried microparticles. In some examples, a tapped density of the combined API hyaluronidase microparticles may be greater than or equal to 0.4 g/mL. Spray drying may be particularly suited to forming microparticles having a tapped density greater than or equal to 0.4 g/mL. In some examples a weight percent of the combined API hyaluronidase microparticles in suspension in the hyaluronidase enhanced microparticle formulation may be greater than or equal to 30%.

A second example 102 of the hyaluronidase enhanced microparticle suspension formulation includes non-aqueous vehicle 106, API microparticles 110, and hyaluronidase particles 112. The second example 102 may be referred to as a co-formulated suspension. In some examples, the API microparticles 110 may not include hyaluronidase. In some examples, the API microparticles 110 may be spray dried microparticles. The hyaluronidase particles 112 may be microparticles, nanoparticles or combinations thereof. In addition to hyaluronidase, hyaluronidase particles 112 may include a bulking agent. For example, the bulking agent may be a sugar, a sugar alcohol, carbohydrate, starch, and/or polysaccharide. As a further example, the bulking agent may include, but is not limited to, trehalose, sucrose, mannitol, maltodextrin, and/or beta-cyclodextrin. The bulking agent may also act as a stabilizer. In some examples, the hyaluronidase particles 112 may not include API. The hyaluronidase particles 112 may be spray dried particles. In some examples, hyaluronidase particles may be microparticles or nanoparticles formed by precipitation, crystallization and/or microglassification. In alternate examples, hyaluronidase particles may be formed as precipitates of hyaluronidase from the non-aqueous vehicle 106. In some examples, a tapped density of the hyaluronidase particles 112 may be greater than or equal to 0.4 g/mL. In some examples, tapped density of a mixture of the hyaluronidase particles 112 and API microparticles 110 may be greater than or equal to 0.4 g/mL. The hyaluronidase enhanced microparticle suspension formulation may only include particles which include hyaluronidase and/or API. A weight ratio of API microparticles 110 to hyaluronidase particles 112 may be much greater than one. The hyaluronidase enhanced microparticle suspension formulation may include more API by weight than hyaluronidase.

A third example 104 of the hyaluronidase enhanced microparticle suspension formulation includes non-aqueous vehicle 106, API microparticles 110, and hyaluronidase suspended in non-aqueous vehicle 106. The hyaluronidase may be directly added to the non-aqueous vehicle 106 and may persist either in a dissolved or precipitated state. In some examples the third example 104 may be an emulsion. In the precipitated state, the hyaluronidase may be hyaluronidase particles similar to hyaluronidase particles 112 and may be suspended in the non-aqueous vehicle. Third example 104 may additionally include stabilizers suspended or dissolved in non-aqueous vehicle 106. Alternatively or in addition to, stabilizers or excipients may be co-dissolved in the hyaluronidase solution prior to addition of hyaluronidase to the non-aqueous vehicle. The stabilizers may prevent aggregation or degradation of the hyaluronidase in non-aqueous vehicle 106.

In some examples, the hyaluronidase enhanced microparticle suspension formulation may include much less hyaluronidase than API. For example, a mass ratio of hyaluronidase to API in the hyaluronidase enhanced microparticle suspension formulation may be significantly less than 1. In some examples the mass ratio of hyaluronidase to API may be less than 1:10. In further examples the mass ratio of hyaluronidase may be less than less than 1:50, less than 1:100, less than 1:1000, less than 1:5,000, less than 1:10000, or ranges therebetween. In such examples, the hyaluronidase enhanced microparticle suspension formulation including the combined API hyaluronidase microparticles may improve uniformity of hyaluronidase in the formulation as compared to hyaluronidase microparticle formations including the API microparticles and the hyaluronidase particles.

Additionally, or alternatively, the hyaluronidase enhanced microparticle suspension formulation may include API in the form of microparticles, non-aqueous vehicle, and hyaluronidase as a combination of one or more of the first example 100, second example 102, and third example 104. For example, the hyaluronidase enhanced microparticle suspension formulation may include one or more of API microparticles and combined API hyaluronidase microparticles in addition to one or more of hyaluronidase microparticles and directly suspended hyaluronidase.

As a further example, the hyaluronidase enhanced microparticle suspension formulation may be in the form of a kit 600 as shown in FIG. 5. Kit 600 may include a non-aqueous component 602 and an aqueous component 604. Non-aqueous component 602 may include API microparticles 110 and the non-aqueous vehicle 106. Aqueous component 604 may include hyaluronidase directly dissolved in an aqueous buffer 606. The non-aqueous component 602 and aqueous component 604 may be physically coupled together in kit 600. In this way, kit 600 is configured for injection of the non-aqueous component 602 and aqueous component 604 at the same time or in quick succession.

FIG. 2 shows a method 300 for producing a spray dried microparticle suspension formulation including combined API hyaluronidase microparticles. The spray dried microparticle suspension formulation formed by method 300 may be an example of first example 100 described above with respect to FIG. 1.

At 302, method 300 includes combining an API, hyaluronidase, formulation excipients, and a liquid phase to form a combined spray drying solution. Combining the API, hyaluronidase, formulation excipients and liquid phase may include adding the API, hyaluronidase, and formulation excipients to the liquid phase and mixing until the API, hyaluronidase, and formulation excipients are fully dissolved or otherwise homogeneously dispersed. The API and hyaluronidase may be the same API and hyaluronidase as described above with respect to FIG. 1. The liquid phase may be a solvent or blend of solvents selected to dissolve the API and hyaluronidase and evaporate at a desired temperature and rate within the spray dryer. In some examples, the liquid phase may include or be water. In some examples the combined spray drying solution may include additional spray drying excipients such as stabilizing excipients or surfactants.

At 304, method 300 includes spray drying the combined spray drying solution to form combined API hyaluronidase microparticles (e.g., combined API hyaluronidase microparticles 108). Spray drying may include spray drying at a Tₒᵤₜ below a threshold temperature. Tₒᵤₜ refers to the temperature at an outlet of a spray dryer. The threshold temperature may be a temperature above which the API or hyaluronidase may degrade and become inactive. In some examples, Tₒᵤₜ may be below an atmospheric boiling point of the liquid phase. In some examples, spray drying may include spray drying at pressures below atmospheric pressure. In some examples, concentrations of the combined liquid phase solution and spray drying parameters (e.g., Tₒᵤₜ, pressure) may be selected to increase a tapped density of the combined API hyaluronidase microparticles.

At 305, method 300 includes drying combined API hyaluronidase microparticles to a target moisture content and/or residual solvent level. In some examples drying may include active drying (e.g., applying heat to drive off moisture). Additionally or alternatively, drying may include equilibrating the API hyaluronidase particles to an atmosphere of the desired moisture level. In some examples, the dried particles may be stored and optionally transported before being reconstituted for use at step 306.

At 306, method 300 includes mixing the combined API hyaluronidase microparticles and a non-aqueous vehicle to form the hyaluronidase enhanced microparticle suspension formulation. The non-aqueous vehicle may be non-aqueous vehicle 106 as described above. Mixing may include agitating the combined hyaluronidase microparticles in the non-aqueous vehicle to form a homogeneous suspension. A concentration of API in the hyaluronidase enhanced microparticle formulation may be greater than or equal to 150 mg/mL. In some examples, mixing may also include mixing additional formulation excipients together with the combined API hyaluronidase microparticles.

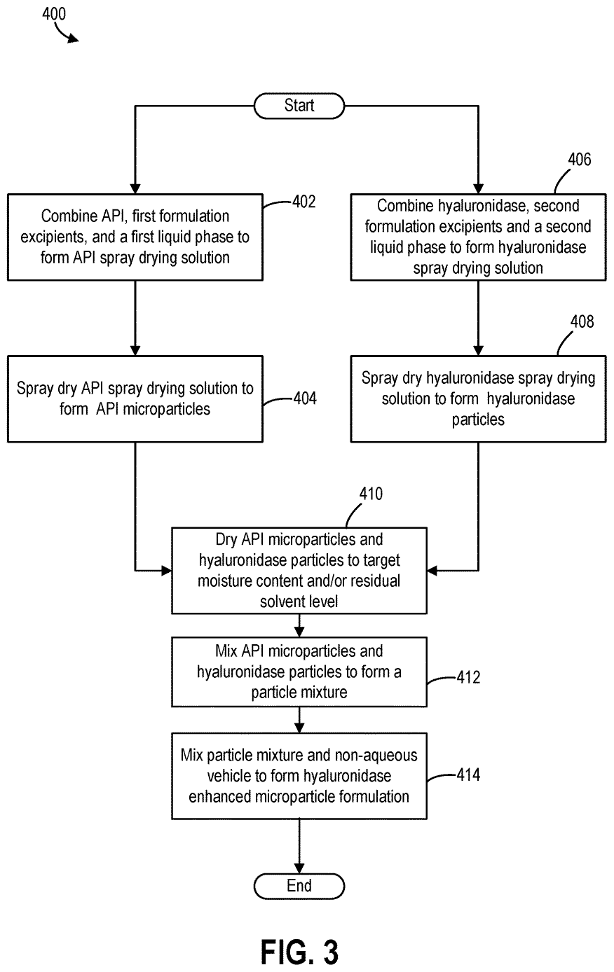

FIG. 3, shows a method 400 for producing a spray dried microparticle suspension formulation including spray dried API microparticles and hyaluronidase particles. The spray dried microparticle formulation formed by method 400 may be an example of second example 102 shown described above with respect to FIG. 1

At 402, method 400 includes combining API, first formulation excipients, and a first liquid phase to form an API spray drying solution. The first liquid phase may be a solvent and or blend of solvents selected to dissolve the API and to evaporate a desired temperature and rate within a spray dryer. In some examples the solvent may be water. The first formulation excipients may include stability excipients to increase stability of the API during spray drying or to increase shelf-life stability of the spray dried powder or subsequent drug product. In one example, the API spray drying solution may not include hyaluronidase.

At 404, method 400 includes spray drying the API spray drying solution to from API microparticles. Spray drying may proceed similar to the spray drying as described above with respect to step 304 of method 300. The threshold temperature for spray drying the API spray drying solution may be a temperature above which the API may be degraded.

At 406, method 400 includes combining hyaluronidase, second formulation excipients, and a second liquid phase to form a hyaluronidase spray drying solution. The second liquid phase may include the same or different solvents than those included in the first liquid phase used to form the API spray drying solution. In some examples the liquid phase may be aqueous. The second formulation excipients may include excipients such as stabilizing excipients and bulking excipients. In on example, the hyaluronidase spray drying solution may not include an API.

At 406, method 400 includes spray drying the hyaluronidase spray drying solution to form hyaluronidase particles. Spray drying may proceed similar to the spray drying as described above with respect to step 304 of method 300. The threshold temperature for spray drying the hyaluronidase may be a temperature above which the hyaluronidase may degrade.

Steps 402 and 404 may be performed in parallel with steps 406 and 408. In alternate examples, steps 402 and 404 may be performed before or after steps 406 and 408. After both API microparticles and hyaluronidase particles are formed, method 400 proceeds to 410 and includes drying the API microparticles and hyaluronidase particles to a target moisture content and/or residual solvent level. Drying may be similar to drying at step 305 of method 300.

At 412, method 400 includes mixing API microparticles and hyaluronidase particles to form a particle mixture. Mixing may include blending the API microparticles and hyaluronidase particles until the API microparticles and hyaluronidase particles are distributed homogeneously. In this way, the particle mixture may be a homogeneous mixture with API microparticles and hyaluronidase particles evenly distributed throughout. In some examples, the particle mixture and/or separate dried API microparticles and hyaluronidase particles may be stored and optionally transported before reconstitution at step 414.

At 414, method 400 includes mixing the particle mixture and a non-aqueous vehicle to from the hyaluronidase enhanced microparticle suspension formulation. Mixing may occur similarly to mixing at step 306 of method 300.

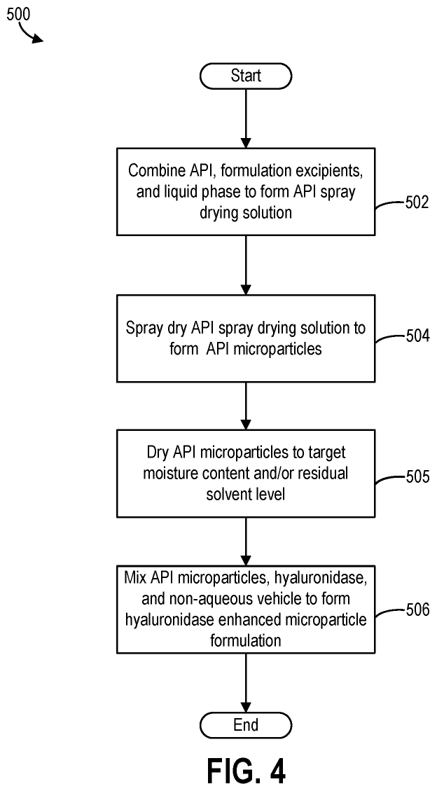

FIG. 4, shows a method 500 for producing a spray dried microparticle suspension formulation including API microparticles and hyaluronidase. The spray dried microparticle suspension formulation formed by method 500 may be an example of third example 104 shown described above with respect to FIG. 1

At 502, method 500 includes combining the API, formulation excipients, and a liquid phase to form an API spray drying solution. Step 502 may be the same as step 402 of method 400. At 504, method 500 includes spray drying the API spray drying solution to form API microparticles. Step 504 may be the same as step 404 of method 400.

At 505, method 500 includes drying the API microparticles to a target moisture content and/or residual solvent level. Drying may be performed similarly to drying as described above with respect to step 305 of method 300.

At 506, method 500 includes mixing the API microparticles, hyaluronidase, and non-aqueous vehicle to form the hyaluronidase enabled microparticle formulation. Mixing may include agitating the API microparticles and hyaluronidase in the non-aqueous vehicle to form a homogenous suspension. Step 506 may, in some examples, include mixing a stabilizing agent along with the API microparticles, hyaluronidase and non-aqueous vehicle. In some examples, mixing may cause the hyaluronidase to precipitate from the non-aqueous vehicle, thereby forming hyaluronidase particles. In this way, mixing at 506, may in some examples, form a formulation similar to the second example 102 shown in FIG. 1. In some examples, step 506 may occur shortly before an injection of the hyaluronidase enabled microparticle suspension formulation is administered.

### Example 1: Co-Processed Hyaluronidase & mAb SDP

As shown in first example 100, the hyaluronidase may be co-formulated in the microparticle with the API to form combined API hyaluronidase microparticles 108. In this example the formulation strategy may have advantages due to synergy in the manufacturing processes and formulations that ensure stability and activity for both the enzyme and therapeutic protein. In this example, the strategy may have advantages compared to manufacture of two separate spray dried particles (SDPs), one containing mAb, or other API, and one containing enzyme, as the strategy demands a single lot of SDP to be manufactured. Additionally, In this example, the approach may reduce the risk to content uniformity and enzyme potency that may be encountered due to the drastically different doses on a mass basis that are typically demanded in a mAb and hyaluronidase combination product.

The following example demonstrates the manufacture of an SDP comprised of mAb and hyaluronidase at mass ratios that are therapeutically relevant to mAb/hyaluronidase combination products. In other examples, different API and ratios may be considered. The SDP was manufactured under process conditions and in a formulation that was previously identified as optimal for bevacizumab, the model antibody used in this example. In this example, the successful integration of hyaluronidase into this formulation and process in combination with bevacizumab was demonstrated in comparable enzymatic activity in the SDP and the starting enzyme. As SDP properties are known to be directly related to performance in high concentration suspensions, a comparison of the SDP properties for bevacizumab SDP produced with the same formulation and process with and without hyaluronidase further highlights the suitability of co-processing hyaluronidase with mAb into a spray dried powder.

Table 1 below describes the solution composition that was spray dried to produce the co-processed mAb/enzyme SDP particles, as an example of method 300 described above. FIG. 6 and compare hyaluronidase activity in the enzyme starting material to hyaluronidase activity in the reconstituted SDP. Enzyme active is measured according to the enzymatic assay of hyaluronidase by turbidimetric determination as described by MilliporeSigma in section (3.2.1.35) of the enzymatic activity assay section of the MilliporeSigma website (https://www.sigmaaldrich.com/US/en/technical-documents/protocol/protein-biology/enzyme-activity-assays/enzymatic-assay-of-hyaluronidase?srsltid=AfmBOooAkptUJn1IXUKP-S_2x6IHEjimzshHieO2WQiT5g0wzuaX2wO2, Accessed Feb. 26, 2026).

TABLE 1
Solution formulation for co-processed hyaluronidase
and bevacizumab. The described solution was spray
dried to produce a co-processed spray dried powder.

| Component | Solution Concentration | |
|---|---|---|
| Bevacizumab | 28 | mg/mL |
| Trehalose | 9.33 | mg/mL |
| Polysorbate 20 | 0.05 | mg/mL |
| Sodium Phosphate | 50 | mM |
| Hyaluronidase | 280 | U/mL |

FIG. 6 shows a chart 700 comparing a percent activity relative to hyaluronidase starting material of a hyaluronidase starting material and hyaluronidase in reconstituted, co-processed SDPs. Column 702 corresponds to the hyaluronidase starting material. Column 704 corresponds to hyaluronidase in co-processed spray dried particles. The co-processed spray dried particles may be an example of combined API hyaluronidase particles 108. As shown in chart 700 and Table 2 below, after reconstitution the hyaluronidase of the co-processed spray dried particle has substantially the same activity as before being processed into a spray dried particle.

TABLE 2
Hyaluronidase activity in reconstituted co-processed SDP. Samples
were analyzed in triplicate according to the Millipore Sigma protocol
described in Enzymatic Assay of Hyaluronidase (3.2.1.35)

| | Activity Relative | % |
|---|---|---|
| Test | to Starting Material | RSD |
| Hyaluronidase Starting Material | 100% | 10% |
| Hyaluronidase in Reconstituted, | 101% | 8% |
| Co-Processed SDP | | |

### Example 2: Hyaluronidase SDP

As an alternative approach to incorporating hyaluronidase into a high concentration suspension, hyaluronidase may be formulated in a microparticle as the only active component, as shown in second example 102 of FIG. 1. In this example, the resulting microparticle may then be either reconstituted for administration or suspended in a vehicle for administration as a suspension. As a solution, hyaluronidase is available on its own and co-formulated with APIs for co-administration. On its own, hyaluronidase products may be administered to the injection site just prior to administration of the active solution. In such examples, the independent administration of these products may allow for broader application and use of hyaluronidase with existing products without the demand for development and production of a combination product. This also allows for independent adjustment of the hyaluronidase and API at the time of administration without being limited by a fixed dose combination. For example, hyaluronidase delivered as a suspension may provide the same value and may be combined in administration with the delivery of products that are either solutions or suspensions.

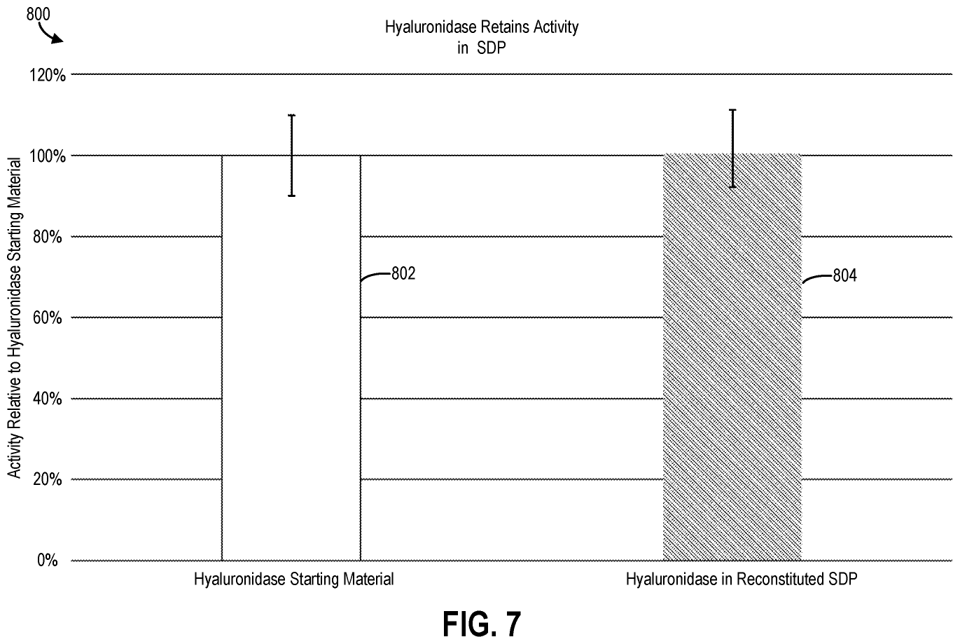

shows the solution formulation that was spray dried to produce a hyaluronidase SDP as an example of method 400. FIG. 7 and illustrate the activity of hyaluronidase in the SDP compared with the starting material.

TABLE 3
Solution formulation spray dried to produce hyaluronidase SDP

| Component | Hyaluronidase Solution Concentration | |
|---|---|---|
| Trehalose | 48.55 | mg/mL |
| Polysorbate 20 | 0.55 | mg/mL |
| Buffer | 50 mM sodium acetate, pH 5.0 | |
| Hyaluronidase | 2182 | U/mL |

TABLE 4
Hyaluronidase activity in reconstituted SDP. Samples were analyzed
in triplicate according to the Millipore Sigma protocol described
in Enzymatic Assay of Hyaluronidase (3.2.1.35) and compared to
the activity of the hyaluronidase starting material.

| | Activity Relative | % |
|---|---|---|
| Test | to Starting Material | RSD |
| Hyaluronidase Starting Material | 100% | 10% |
| Hyaluronidase in Reconstituted SDP | 100% | 11% |

FIG. 7 shows a chart 800 comparing hyaluronidase activity relative to starting material for hyaluronidase starting material to hyaluronidase which was spray dried into a particle and then reconstituted. The spray dried hyaluronidase is an example of hyaluronidase particle 112 as described above with respect to FIG. 1. Column 802 corresponds to the hyaluronidase starting material and column 804 corresponds to the spray dried and reconstituted hyaluronidase particles. As shown in FIG. 7 and Table 4, activity of the hyaluronidase is substantially the same before and after spray drying.

### Example 3: Hyaluronidase SDP Co-Formulated with SDP Containing API

As another non-limiting example, the hyaluronidase microparticle may be co-formulated with another active pharmaceutical ingredient through either physical mixing of two solid state powders (for example, two spray dried powders) or through combining the two powders in suspension. In such examples, the benefit in the co-formulation approach to co-delivery of hyaluronidase with an active pharmaceutical ingredient when compared with co-processing is the flexibility in formulating to meet the specific demands of the hyaluronidase and active pharmaceutical ingredient independently. This is illustrated in the example provided below in which the hyaluronidase SDP is manufactured from an example of hyaluronidase spray drying solution, including sodium acetate buffer at pH 5.0 and the bevacizumab SDP is manufactured from an example of an API spray drying solution including sodium phosphate buffer at pH 6.2. Example compositions of the bevacizumab spray drying solution and hyaluronidase spray drying solution are provided in Table 5 below. Hyaluronidase is more stable at pH 5.0 than at a pH 6.2 and bevacizumab is more stable at pH 6.2 than at pH 5.0, and independent SDP formulations allow for independent optimization of each formulation. In alternate examples, other buffer compositions and target pHs may be used for the hyaluronidase spray drying solutions and for the API spray drying solution.

An additional benefit to this approach is the flexibility in independently adjusting the hyaluronidase and active ingredient dose, allowing for a single lot of hyaluronidase SDP and a single lot of SDP containing an API to be leveraged to produce multiple final products or test articles with varying ratios of hyaluronidase and API. The formulation of the hyaluronidase SDP may be developed to optimize hyaluronidase activity and stability, as previously described, enabling downstream processing that is suitable for the final drug product. As the target doses of hyaluronidase are relatively low on a mass basis, a bulking agent may be used to enable powder handling in downstream blending or filling operations or dose uniformity in suspension preparation and fill/finish activities, particularly in co-formulations. For co-formulation in suspension formulations aimed at delivering high doses of API, the bulking agent concentration may be minimized to achieve enzyme stability and content uniformity without impacting the deliverable dose of API which is limited by the total microparticle content in suspension regardless of which active component the microparticle contains. The bulking agent may be selected to optimize powder properties and/or it may also act as a stabilizing excipient such as trehalose in the example given here.

TABLE 5
Solution formulations spray dried
to produce SDP for co-formulation.

| | Bevacizumab Solution | Hyaluronidase Solution | |
|---|---|---|---|
| Component | Concentration | Concentration | |
| Bevacizumab | 90 mg/mL | NA | |
| Sucrose | 30 mg/mL | NA | |
| Trehalose | NA | 48.55 | mg/mL |
| Polysorbate 20 | 0.4 mg/mL | 0.55 | mg/mL |

| | | |
|---|---|---|
| Buffer | 50 mM sodium | 50 mM sodium |
| | phosphate, pH 6.2 | acetate, pH 5.0 |
| Hyaluronidase | NA | 39.0 |

### Example 4: Hyaluronidase Delivered in Non-Aqueous Suspensions

Co-administration of hyaluronidase in suspension with an active pharmaceutical ingredient such as a monoclonal antibody may be achieved through co-processing, co-formulation, or direct addition of hyaluronidase solution to the suspension vehicle. To evaluate the activity of hyaluronidase in each of these product presentations, suspensions were prepared and reconstituted in a manner to mimic dissolution upon administration, and hyaluronidase activity was measured in the resulting solution. The following suspensions were prepared and evaluated:
Co-processed hyaluronidase and bevacizumab SDP was suspended in Miglyol® 840 at about 50 wt % to target a mAb concentration of around 350 mg/mL and a hyaluronidase activity of approximately 6400 U/mL.
To produce a co-formulated suspension, hyaluronidase microparticles and bevacizumab microparticles produced from spray drying the solutions described in Table 5 were blended and suspended in Miglyol® 840 to target approximately 250 mg/mL bevacizumab and 6800 U/mL hyaluronidase.
To produce a suspension comprised of bevacizumab SDP and a hyaluronidase emulsion, hyaluronidase stock solution containing 63.6 kU/mL hyaluronidase was added to Miglyol® 840 to target a final hyaluronidase concentration of 6688 U/mL and vigorously mixed to disperse the aqueous phase. Bevacizumab microparticles were then added to the resulting emulsion at a target of approximately 50 wt % to target a final mAb concentration of 330 mg/mL.

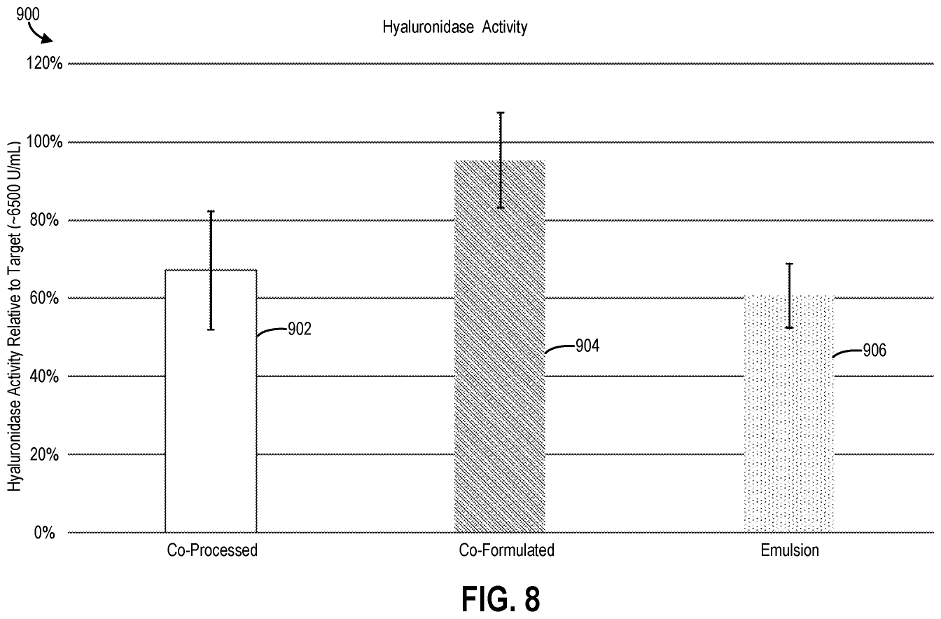

FIG. 8 shows a chart 800 which compares the measured hyaluronidase activity relative to the target in each suspension listed above. A first column 902 corresponds to the co-processed suspension listed above. A second column 904 corresponds to the co-formulated suspension listed above. FIG. 8 further compares a formulation in which hyaluronidase solution is co-formulated in the suspension vehicle with the solid-state protein microparticles. The formulation may be referred to as an emulsion and may be an example of a formulation formed from a kit, as described above with respect to FIG. 6. A third column 906 corresponds to the emulsion listed above.

Commercially available technologies that co-formulate or co-deliver hyaluronidase with protein solutions to increase the deliverable volume, decrease the injection time, and overall improve patient experience and even, in some cases, improve exposure leverage formulations of hyaluronidase in solution. In some instances, hyaluronidase may be formulated in a separate solution than the API to be delivered to the same injection site just prior to administration of the therapeutic solution. This might be referred to as co-delivery of hyaluronidase with a therapeutic. However, this co-delivery is simplified by the fact that both the protein and hyaluronidase are in solution and does not demand co-formulation of hyaluronidase with the therapeutic. In contrast, the approach outlined in the examples above may be applied to delivery of a suspension formulation containing the API. As hyaluronidase is typically formulated as an aqueous solution, additional steps as described above were used to provide a co-formulation in suspension. The effectiveness of a co-formulation of the hyaluronidase with an API formulation comprised of solid state protein microparticles in a non-aqueous vehicle is surprising.

In other examples and descriptions contained within this document, hyaluronidase may be incorporated into these formulations by co-formulation or co-processing of hyaluronidase incorporated into a solid state microparticle prior to suspension in a non-aqueous vehicle. In this example, the incorporation of hyaluronidase directly into the suspension vehicle is considered.

As shown in FIG. 8, each of the suspensions show hyaluronidase activity, suggesting that delivery of hyaluronidase in suspension to act on the extracellular matrix when delivered subcutaneously can be achieved. The lower than target activity of hyaluronidase in the co-processed and emulsion formulations compared to the co-formulated suspension suggests that optimization of the microparticle formulation to improve hyaluronidase stability may provide benefit.

In the examples shown in FIG. 8, Miglyol® 840 was used as a suspension vehicle that is known to be compatible with protein SDPs. Other vehicles with known compatibility may include medium chain triglycerides, triacetin, sesame oil, Captex® 355, Miglyol® 812N, and ethyl acetate.

The technical effect of the methods 300, 400, and 500, is to form a hyaluronidase enhanced microparticle suspension formulation. The hyaluronidase enhanced microparticle suspension formulations combine the benefits of a non-aqueous suspension formulation with added hyaluronidase. The hyaluronidase enhanced microparticle suspension formulation may be preferred to conventional microparticle formulations which do not include hyaluronidase due to an increase rate of dispersion of the injected suspension in the subcutaneous space and potential subsequent increased bioavailability of the API enabled by the inclusion of hyaluronidase in the non-aqueous suspension.

The disclosure also provides support for a hyaluronidase enhanced microparticle suspension formulation, comprising: microparticles including an active pharmaceutical ingredient (API), hyaluronidase, and a non-aqueous vehicle. In a first example of the system, the microparticles are spray dried microparticles. In a second example of the system, optionally including the first example, the microparticles include the hyaluronidase and are combined API hyaluronidase microparticles. In a third example of the system, optionally including one or both of the first and second examples, the hyaluronidase is included in hyaluronidase particles. In a fourth example of the system, optionally including one or more or each of the first through third examples, the hyaluronidase particles are microparticles or nanoparticles. In a fifth example of the system, optionally including one or more or each of the first through fourth examples, the hyaluronidase is directly suspended in the non-aqueous vehicle. In a sixth example of the system, optionally including one or more or each of the first through fifth examples, a concentration of API in the hyaluronidase enhanced microparticle formulation is greater than or equal to 150 mg/mL. In a seventh example of the system, optionally including one or more or each of the first through sixth examples, a mass ratio of hyaluronidase to API in the hyaluronidase enhanced microparticle formulation is less than 1. In an eighth example of the system, optionally including one or more or each of the first through seventh examples, a tapped density of the microparticles is greater than or equal to 0.4 g/mL. In a ninth example of the system, optionally including one or more or each of the first through eighth examples, the API is a biologic, and wherein the microparticles including the API are not in solution in the non-aqueous vehicle. In a tenth example of the system, optionally including one or more or each of the first through ninth examples, the microparticles are included in a non-aqueous component of a kit and the hyaluronidase is included in an aqueous component of the kit.

The disclosure also provides support for a method for producing a hyaluronidase enhanced microparticle suspenion formulation, comprising: combining an active pharmaceutical ingredient, hyaluronidase, and a liquid phase to form a combined spray drying solution, spray drying the combined spray drying solution to form combined API hyaluronidase microparticles. In a first example of the method, the method further comprises: mixing the combined API hyaluronidase microparticles and a non-aqueous vehicle to form the hyaluronidase enhanced microparticle formulation. In a second example of the method, optionally including the first example, spray drying includes spray drying at a Tout below a threshold temperature, wherein the threshold temperature is a temperature above which the API or the hyaluronidase degrades. In a third example of the method, optionally including one or both of the first and second examples, spray drying includes spray drying at a pressure below atmospheric pressure. In a fourth example of the method, optionally including one or more or each of the first through third examples, a tapped density of the combined API hyaluronidase microparticles is greater than or equal to 0.4 g/mL.

The disclosure also provides support for dried particles for a non-aqueous formulation, comprising: hyaluronidase, and an active pharmaceutical ingredient. In a first example of the system, a dried particle of the dried particles includes both the active pharmaceutical ingredient and the hyaluronidase. In a second example of the system, optionally including the first example, a first dried particle of the dried particles includes hyaluronidase and does not include the active pharmaceutical ingredient, and a second dried particle of the dried particles includes the active pharmaceutical ingredient and does not include hyaluronidase. In a third example of the system, including the second example, the first dried particle further includes a bulking agent.

In an alternate embodiment, the disclosure also provides support for a method for producing a hyaluronidase enhanced microparticle suspension formulation, comprising: combining an active pharmaceutical ingredient (API) and a first liquid phase to form an API spray drying solution, spray drying the API spray drying solution to form API microparticles, and mixing the API microparticles, hyaluronidase, and a non-aqueous vehicle to form the hyaluronidase enhanced microparticle formulation. In a first example of the method, the hyaluronidase is in the form of hyaluronidase particles formed by: combining the hyaluronidase and a second liquid phase to form a hyaluronidase spray drying solution, and spray drying the hyaluronidase to form hyaluronidase particles. In a second example of the method, optionally including the first example, the hyaluronidase particles are microparticles or nanoparticles. In a third example of the method, optionally including one or both of the first and second examples, a tapped density of the hyaluronidase particles is greater than or equal to 0.4 g/mL. In a fourth example of the method, optionally including one or more or each of the first through third examples, wherein the hyaluronidase is added directly to the non-aqueous vehicle.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A hyaluronidase enhanced microparticle suspension formulation, comprising:
microparticles including an active pharmaceutical ingredient (API);
hyaluronidase; and
a non-aqueous vehicle.

2. The hyaluronidase enhanced microparticle suspension formulation of claim 1, wherein the microparticles are spray dried microparticles.

3. The hyaluronidase enhanced microparticle suspension formulation of claim 1, wherein the microparticles include the hyaluronidase and are combined API hyaluronidase microparticles.

4. The hyaluronidase enhanced microparticle suspension formulation of claim 1, wherein the hyaluronidase is included in hyaluronidase particles.

5. The hyaluronidase enhanced microparticle suspension formulation of claim 4, wherein the hyaluronidase particles are microparticles or nanoparticles.

6. The hyaluronidase enhanced microparticle suspension formulation of claim 1, wherein the hyaluronidase is directly suspended in the non-aqueous vehicle.

7. The hyaluronidase enhanced microparticle suspension formulation of claim 1, wherein a concentration of API in the hyaluronidase enhanced microparticle suspension formulation is greater than or equal to 150 mg/mL.

8. The hyaluronidase enhanced microparticle suspension formulation of claim 1, wherein a mass ratio of hyaluronidase to API in the hyaluronidase enhanced microparticle formulation is less than 1.

9. The hyaluronidase enhanced microparticle suspension formulation of claim 1, wherein a tapped density of the microparticles is greater than or equal to 0.4 g/mL.

10. The hyaluronidase enhanced microparticle suspension formulation of claim 1, wherein the API is a biologic, and wherein the microparticles including the API are not in solution in the non-aqueous vehicle.

11. The hyaluronidase enhanced microparticle suspension formulation of claim 1, wherein the microparticles are included in a non-aqueous component of a kit and the hyaluronidase is included in an aqueous component of the kit.

12. A method for producing a hyaluronidase enhanced microparticle suspension formulation, comprising:
combining an active pharmaceutical ingredient (API), hyaluronidase, and a liquid phase to form a combined spray drying solution;
spray drying the combined spray drying solution to form combined API hyaluronidase microparticles.

13. The method of claim 12, further comprising mixing the combined API hyaluronidase microparticles and a non-aqueous vehicle to form the hyaluronidase enhanced microparticle formulation.

14. The method of claim 12, wherein spray drying includes spray drying at a Tₒᵤₜ below a threshold temperature, wherein the threshold temperature is a temperature above which the API or the hyaluronidase degrades.

15. The method of claim 12, wherein spray drying includes spray drying at a pressure below atmospheric pressure.

16. The method of claim 12, wherein a tapped density of the combined API hyaluronidase microparticles is greater than or equal to 0.4 g/mL.

17. Dried particles for a non-aqueous suspension formulation, comprising:
hyaluronidase; and
an active pharmaceutical ingredient.

18. The dried particles of claim 17, wherein a dried particle of the dried particles includes both the active pharmaceutical ingredient and the hyaluronidase.

19. The dried particles of claim 17, wherein a first dried particle of the dried particles includes hyaluronidase and does not include the active pharmaceutical ingredient and a second dried particle of the dried particles includes the active pharmaceutical ingredient and does not include hyaluronidase.

20. The dried particles of claim 19, wherein the first dried particle further includes a bulking agent.
